(19) United States
(12) Patent Application Publication
(10) Pub. No.: US 2010/0023116 A1
(43) Pub. Date: Jan. 28, 2010

(54) BIOCORRODIBLE IMPLANT WITH A COATING CONTAINING A DRUG ELUTING POLYMER MATRIX

(76) Inventors: Alexander Borck, Aurachtal (DE); Nina Adden, Nuemberg (DE)

Correspondence Address: GREER, BURNS & CRAIN, 300 S WACKER DR, 25TH FLOOR, CHICAGO, IL, 60606, US

(21) Appl. No.: 12/509,831

(22) Filed: Jul. 27, 2009

(30) Foreign Application Priority Data
Jul. 28, 2008 (DE) 10 2008 040 786.0

(51) Int. Cl. A61F 2/06

(52) U.S. Cl. 623/1.42; 623/1.46

(57) ABSTRACT

The invention relates to an implant having a base body, consisting completely or partially of a biocorrodible metallic material, such that it decomposes in an aqueous environment to form an alkaline product, and the base body has a coating or a cavity filling, comprising a polymer matrix and at least one drug embedded in the polymer matrix, characterized in that at least one polymer of the matrix and the at least one drug are coordinated so that the drug elution rate from the matrix is increased with an increase in pH.

### FIELD OF THE INVENTION

The invention relates to a biocorrodible implant with a coating containing a drug eluting polymer matrix.

### BACKGROUND OF THE INVENTION

Implants have gained acceptance in modem medical technology in a variety of embodiments. They serve primarily to support vessels, hollow organs and duct systems (endovascular implants), for fastening and temporary fixation of tissue implants and tissue transplants but also for orthopedic purposes, e.g., as nails, plates or screws.

For example, implantation of stents has become established as one of the most effective therapeutic measures in treatment of vascular diseases. The purpose of stents is to assume a supporting function in a patient's hollow organs. Stents of the traditional design therefore have a filigree supporting structure comprising metallic struts, which are initially in a compressed form for introducing them into the body and then are expanded at the site of application. One of the main fields of application of such stents is for permanent or temporary dilatation and maintaining the patency of vasoconstrictions, in particular stenoses of the coronary vessels. In addition, there are also known aneurysm stents, for example, which serve to support damaged vascular walls.

Stents have a circumferential wall of a sufficient supporting strength to keep the constricted vessel open to the desired extent, and have a tubular base body through which the blood continues to flow unhindered. The circumferential wall is usually formed by a mesh-like supporting structure, allowing the stent to be inserted in a compressed form having a small outside diameter as far the constriction in the respective vessel to be treated, and to widen it there with the help of a balloon catheter, for example, to the extent that the vessel has the desired dilated inside diameter. A cardiologist must monitor the procedure of positioning and expansion of stents and the final position of stent in the tissue after the end of the procedure. This can be accomplished by imaging methods, e.g., by radiology.

The implant or the stent has a base body of an implant material. An implant material is a nonliving material, which is used for an application in medicine and interacts with biological systems. The basic prerequisites for use of a material as an implant material that comes in contact with the physiological environment when used as intended is its biocompatibility. Biocompatibility is understood to be the ability of a material to induce an appropriate tissue reaction in a specific application. This comprises an adaptation of the chemical, physical, biological and morphological surface properties of an implant to the recipient tissue with the goal of achieving a clinically desired interaction. The biocompatibility of the implant material also depends on the time sequence of the reaction of the biosystem in the implant. Thus relatively short-term irritations and inflammations occur and lead to tissue changes. Biological systems thus react differently, depending on the properties of the implant material. According to the reaction of the biosystem, implant materials can be subdivided into bioactive, bioinert and degradable/absorbable materials.

A biological reaction to polymeric, ceramic or metallic implant materials depends on the concentration, duration of exposure and how administered. The presence of an implant material often leads to inflammation reactions triggered by mechanical irritation, chemicals and metabolites. The inflammation process is usually accompanied by migration of neutrophilic granulocytes and monocytes through the vascular walls, migration of lymphocyte effector cells, forming specific antibodies to the inflammation irritant, activation of the complement system and the release of complement factors which act as mediators and ultimately the activation of blood coagulation. An immunologic reaction is usually closely associated with an inflammation reaction and may lead to sensitization and allergization. Known metallic allergens comprise, for example, nickel, chromium and cobalt, which are also used as alloy components in many surgical implants. One important problem in stent implantation in blood vessels is in-stent restenosis due to excessive neointimal growth induced by highly proliferating smooth arterial muscle cells and a chronic inflammation reaction.

One promising approach to solving this problem lies in the use of biocorrodible metals and their alloys as the implant material because a permanent supporting function by the stent is not usually necessary. Initially damaged body tissues regenerate. In DE 197 31 021 A1, for example, it is proposed that medical implants should be made of a metallic material with the main component being iron, zinc or aluminum and/or an element from the group of alkali metals or alkaline earth metals. Alloys based on magnesium, iron and zinc are described as especially suitable. Secondary components of the alloys may include manganese, cobalt, nickel, chromium, copper, cadmium, lead, tin, thorium, zirconium, silver, gold, palladium, platinum, silicon, calcium, lithium, aluminum, zinc and iron. In addition, it is known from DE 102 53 634 A1 that a biocorrodible magnesium alloy with a magnesium content of >90%, yttrium 3.7-5.5%, rare earth metals 1.5-4.4% and the remainder <1% is suitable in particular for production of an endoprosthesis, e.g., in the form of a self-expanding or balloon-expandable stent. Use of biocorrodible metallic materials in implants should lead to a definite reduction in rejection or inflammation reactions. Such biocorrodible implants and stents often have a coating or cavity filling with a suitable polymer.

One problem when using these biocorrodible implants consisting completely or partially of a metallic material is that the degradation products which are formed and eluted in corrosion of the implant often have a significant influence on the local pH and thus can lead to unwanted tissue reactions as well as possibly having an adverse effect on the further corrosion rate of the implant. In degradation of biocorrodible implant materials containing Mg in particular, there may be an increase in the pH in the immediate vicinity. This increase in pH may lead to a phenomenon known by the term alkalosis. The local increase in pH results in an imbalance in charge distribution in muscle cells surrounding the blood vessel, which may lead to a local increase in muscle tone in the area of the implant. This increased pressure on the implant may lead to premature loss of implant integrity.

The object of the present invention was to reduce or overcome one or more of the disadvantages of the prior art described above.

### SUMMARY OF THE INVENTION

This object is achieved by providing an implant with a base body consisting completely or partially of a biocorrodible metallic material. The material is such that it decomposes to an alkaline product in an aqueous environment, and the base body has coating or a cavity filling comprising a polymer matrix and at least one drug embedded in the polymer matrix, characterized in that at least one polymer of the matrix and the at least one drug are coordinated so that the drug elution rate from the matrix is increased at an elevated pH.

One advantage of the inventive approach is that the embedded drugs are eluted from the polymer matrix to an increased extent with a time delay and also with spatial limitations only when a locally elevated pH is prevailing.

When using the inventive implant, it is no longer necessary to counteract a possible alkalosis, for example, by systemic administration of medicines or drugs. Corresponding drugs may already be embedded in the coating of the biocorrodible implant and are eluted to an increased extent at the site when there is a change in the local pH. Thus on the whole, definitely smaller doses of drug may be used, which are then preferably made available at the desired site and at the time of need. The patient is less burdened and treatment costs are reduced.

Implants in the sense of this invention are devices introduced into the body by a surgical procedure and comprise fastening elements for bones, e.g., screws, plates or nails, surgical suture material, intestinal clamps, vascular clips, prostheses in the area of the hard and soft tissue and anchoring elements for electrodes, in particular pacemakers or defibrillators.

### DETAILED DESCRIPTION OF THE INVENTION

The implant is preferably a stent. Stents of the traditional design have a filigree supporting structure of metallic struts, which are initially in an unexpanded state for introduction into the body and then are widened at the site of application into an expanded state. The stent may be coated before or after being crimped onto a balloon.

According to a first variant, the base body of the implant thus has a coating containing or comprising an inventive polymer matrix and at least one drug embedded in the polymer matrix. A coating in the inventive sense is formed when components of the coating are applied in at least some sections to the base body of the implant. The entire surface of the base body of the implant is preferably covered by the coating. The layer thickness is preferably in the range of 1 nm to 100 μm, especially preferably 300 nm to 30 μm. The amount by weight of inventive polymer matrix in the components forming the coating is preferably at least 40%, especially preferably at least 70%. The coating may be applied directly to the implant surface. The processing may then be performed according to standard coating methods. Single-layer systems as well as multilayer systems (e.g., so-called base coat layers, drug coat layers or top coat layers) may also be created. The coating may be applied directly to the base body of the implant or other layers may be provided in between, e.g., to improve adhesion.

As an alternative, the polymer matrix comprising the at least one drug embedded in the polymer matrix may be in the form of a cavity filling or as a component of a cavity filling. The implant, in particular the stent, therefore has one or more cavities. Cavities are provided on the surface of the implant, for example, and may be created with dimensions in the micrometer range, e.g., by laser ablation. In the case of implants, in particular stents with a biodegradable base body, a cavity may also be provided in the interior of the base body, so that the material is eluted only after being exposed. In designing the cavity, those skilled in the art may rely on systems described in the prior art.

Alloys and elements in which degradation and/or conversion occur in a physiological environment are known as biocorrodible in the sense of the present invention, such the part of the implant consisting of the material is entirely or at least predominantly no longer present. The biocorrodible metallic materials in the sense of the invention comprise metals and alloys selected from the group consisting of iron, tungsten, zinc, molybdenum and magnesium and in particular biocorrodible metallic materials which corrode in an aqueous solution to form an alkaline product.

The metallic base body preferably consists of magnesium, a biocorrodible magnesium alloy, pure iron, a biocorrodible iron alloy, a biocorrodible tungsten alloy, a biocorrodible zinc alloy or a biocorrodible molybdenum alloy. The biocorrodible metallic material is a magnesium alloy in particular.

A biocorrodible magnesium alloy is understood to be a metallic structure having magnesium as its main component. The main component is the alloy component present in the greatest amount by weight of the alloy. The amount of main component is preferably more than 50 wt %, in particular more than 70 wt %. The biocorrodible magnesium alloy preferably contains yttrium and other rare earth metals because such an alloy is characterized by its physicochemical properties and high biocompatibility, in particular also its degradation products. An especially preferred magnesium alloy has a composition comprising 5.2-9.9 wt % rare earth metals, including 3.7-5.5 wt % yttrium and <1 wt % remainder, where magnesium accounts for the rest of the alloy up to 100 wt %. This magnesium alloy has already confirmed its special suitability experimentally and in preliminary clinical experiments, i.e., it has a high biocompatibility, favorable processing properties, good mechanical characteristics and an adequate corrosion behavior for the intended purpose. In the present case, the collective term “rare earth metals” is understood to include scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71).

The composition of the magnesium alloy is to be selected so that it is biocorrodible. Artificial plasma such as that specified according to EN ISO 10993-15:2000 for biocorrosion investigations (composition: NaCl 6.8 g/L, CaCl₂ 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L) is used as the test medium for testing the corrosion behavior of alloys. A sample of the material to be tested is stored in a defined amount of the test medium at 37° C. in a sealed sample container. At intervals of time—coordinated with the expected corrosion behavior—of a few hours up to several months, samples are taken and tested for traces of corrosion in a known way. The artificial plasma according to EN ISO 10993-15:2000 corresponds to a blood-like medium and offers a possibility for reproducibly simulating a physiological environment in the sense of this invention.

According to the invention, the at least one polymer of the polymer matrix and the at least one drug are coordinated so that the drug elution rate from the polymer matrix is increased at an elevated pH.

Such an elevated pH occurs when the local pH is shifted to the basic pH range in comparison with the physiological pH. An elevated pH in the sense of the present invention in particular prevails when the pH in the local environment is greater than 8.

The term “elution rate” in the sense of the invention is understood to be the amount of drug eluted from the polymer matrix per unit of time. Those skilled in the art know of suitable processes and measurement methods for determining elution rate. Such measurement methods as those that have already repeatedly proven successful in the field of galenics in particular are suitable.

In a preferred embodiment, the drug elution rate from the polymer matrix is elevated at a pH above 8. In especially preferred embodiments, the drug elution rate from the polymer matrix is increased by a factor of at least 2 when the pH is higher than 8 in comparison with the elution rate at a physiological pH.

The at least one polymer of the polymer matrix may have pH-dependent properties. The polymer has, for example, at least one functional group which shows a transition between a neutral charge state and an ionic charge state with an increase in pH. The at least one functional group of the polymer may be in an ionic charge state at physiological pH and may be in a neutral charge state at an elevated pH in the sense of the present invention. It is also possible for the at least one functional group to be in a neutral state at a physiological pH and to be in an ionic charge state at an elevated pH. In such a system, the drug may be eluted to a greater extent by shifting of charge states in the polymer matrix in transition from a physiological pH to an elevated pH. The at least one polymer may have several functional groups that are the same or different and need not all be in the same charge state at a physiological pH.

In a preferred embodiment, the at least one functional group is selected from the group comprising a carboxylic acid function, an amine function or an amide function.

In one embodiment of the invention, the polymer matrix comprises a hydrogel. A hydrogel is a polymer that contains water but is insoluble in water, its molecules being bonded chemically, by covalent or ionic bonds or physically, e.g., by linking of polymer chains to form a three-dimensional network. Inventive hydrogels are capable of changing their volume when there is a change in pH by either taking up more water with an increase in volume when the pH is elevated or taking up less water with a decline in volume. These hydrogels can be produced, for example, by reaction of ethylenically unsaturated monomers and polymers having ionizable groups with crosslinking agents and polymerization catalysts. As an alternative to that, suitable hydrogels can also be prepared by condensation reactions with difunctional and polyfunctional monomers. Those skilled in the art know of suitable monomers and polymers as well as methods of producing them. Those skilled in the art know of methods and processes for producing suitable hydrogels by means of such monomers and/or polymers. Hydrogels expand by an increase in volume at an elevated pH, e.g., when the hydrogel contains carboxyl groups. A reduction in volume of the hydrogel at an elevated pH may occur, for example, when the network contains amine groups and/or amide groups.

Preferred hydrogels contain a polymer based on acrylic acid, methacrylic acid or a derivative of acrylic acid or methacrylic acid.

According to the invention, the drug may be embedded in the polymer matrix as a prodrug, for example. The drug is initially coupled to macromolecules which keep the drug embedded in the polymer matrix. Those skilled in the art are aware of such macromolecules and in particular the macromolecule may be dextran. The monomer or polymer of the polymer matrix may be such a macromolecule in the sense of the invention. The prodrug system is characterized in that the drug, coupled by chemical bonds to the macromolecule, is eluted from the polymer matrix by an elevated pH. The chemical bonds attaching the drug to the molecule are then broken and the drug can escape from the polymer matrix. The drug is preferably affixed in the polymer matrix by chemical bonds which can be cleaved in a base-catalyzed process, especially preferably by ester bonds, e.g., sulfonic acid esters, or amide bonds. An example of a suitable prodrug system is given below:

macromolecule-PhSO₂Cl+bosentan-OH→macromolecule-PhSO₂O-bosentan

The reverse reaction, eluting the drug bosentan, then takes place in the presence of hydroxide ions at an elevated pH.

A prodrug system in the sense of the invention is when the drug is present first in encapsulated form, capsules carrying the drug being embedded in the polymer matrix. The drug is then eluted from the capsule and from the polymer matrix to a greater extent at an elevated pH. Those skilled in the art know in particular of suitable formulations of such encapsulations from the field of galenics, for example.

Any known drug that interacts with the polymer matrix of the coating or cavity filling of the implant such that the drug elution rate is increased at an elevated pH may be used as the drug. Drugs suitable for treatment or prevention of alkalosis are preferred. Such drugs are selected from the group comprising vasodilators, anti-inflammatories and local pH regulating drugs. Especially preferred drugs are selected from the group comprising NO-eluting substances and bosentan, dipyridamol, dODN or in general endothelin receptor antagonists, calcium channel blockers such as amlodipine, nifidipine or verapamil.

The inventive implant may have an additional outer coating. Such an additional outer coating may completely or partially cover the coating or cavity filling comprising a polymer matrix and at least one drug. This outer coating may contain or comprise a degradable polymer, in particular a polymer from the class of PLGA (poly(lactic-co-glycolic acid)) or PLGA-PEG block copolymers. A drug that can elute freely or is eluted in degradation of the outer coating may optionally be embedded in this additional outer layer.

Such an additional outer coating may be used to delay the release of the at least one drug from the polymer matrix which is mediated by the change in pH in a multilayer system. The additional outer coating is degraded first and only then does the inner coating become accessible, then eluting the at least one drug in the presence of an elevated pH.

The invention is explained in greater detail below on the basis of exemplary embodiments.

### Exemplary Embodiment 1

### Polyacrylic Acid with Bosentan

5.0 g (69 mmol) acrylic acid is dissolved in 100 mL water at room temperature and degassed with N₂ while stirring for 30 minutes. Polymerization is initiated by adding 1 mol % 2,2′-azobis(2-amidinopropane)dihydrochloride and heating to 60° C. Polymerization is then performed for 12 hours. After cooling to room temperature, the viscous solution is dialyzed against water (molecular cutoff (MCO) 13,000 Da). The swelling capacity of the resulting polyacrylic acid in an aqueous environment increases with an increase in pH.

Matrix preparation and incorporation of drug:

1 g of the resulting polymer is mixed with 30% bosentan.

### Exemplary Embodiment 2

### Poly(N-isopropylacrylamide-co-allylamine) with Verapamil

3.8 g (33.6 mmol) N-isopropylacrylamide (NIPAM) and 0.2 g (3.4 mmol) allylamine (10% of the NIPAM monomer) are dissolved in 230 mL THF (tetrahydrofuran) at room temperature. Then 0.06% SDS and 0.067 g (1.3 mol %; 0.44 mmol) N,N′-methylene-bis-acrylamide are added. The solution is degassed with N₂ for 30 minutes while stirring and heated to 70° C. 0.166 g potassium persulfate is dissolved in 20 mL water and added to the reaction mixture to initiate the reaction. The reaction is performed for 4 hours at 68-70° C. After cooling to room temperature, the precipitate is dialyzed for five days against water (molecular cutoff (MCO) 13,000 Da). The resulting poly(N-isopropylacrylamide-co-allyl-amine) has a reduced swelling ability in an aqueous environment with an increase in pH.

Matrix preparation and incorporation of drug:

1 g of the resulting polymer is mixed with verapamil and crosslinked with 0.04 g (25 wt %)

glutaraldehyde for 2 hours at room temperature.

Alternatively, matrix preparation and embedding of the drug may be performed as follows: 1 g of the resulting polymer is mixed with approx. 300 mg verapamil. Then 0.032 g (0.17 mmol) 1-ethyl-3-(3-dimethylaminopropyl)carbodiimide dissolved in 200 μL water and 0.015 g (0.085 mmol) adipic acid dihydrazide also dissolved in 200 μL water are added and stirred for 2 hours.

### Exemplary Embodiment 3

### Coating a Stent

A stent of the biocorrodible magnesium alloy WE43 (4 wt % yttrium, 3 wt % rare earth metals not including yttrium, remainder magnesium and impurities due to the production process) is coated as follows:

The stent is cleaned of dirt and residues and clamped in a suitable stent coating apparatus (DES coater, in-house development of Biotronik). With the help of an airbrush system (EFD or spraying system companies), the rotating stent is coated on one half side with one of the polymer mixtures from exemplary embodiments 1 or 2 under constant ambient conditions (room temperature, 42% atmospheric humidity). At a nozzle spacing of 20 mm, an 18-mm-long stent is coated after approx. 10 minutes. After reaching the intended layer weight, the stent is dried for 5 minutes at room temperature before the uncoated side is coated in the same way after renewed rotation of the stent and renewed clamping. The finished coated stent is dried for 36 hours at 40° C. in a vacuum oven (Vakucell, MMM). The layer thickness of the applied coating is approx. 10 μm.

### Exemplary Embodiment 4

### Multilayer System

A stent of biocorrodible magnesium alloy WE43 (4 wt % yttrium, 3 wt % rare earth metals not including yttrium, remainder magnesium and impurities due to the production process) is coated first with a solution of high-molecular PLLA (poly-L-lactide) (Boehringer Ingelheim, Mw 300,000) and bosentan (9:1) in chloroform. This stent is therefore cleaned to remove dust and residues and is clamped in a suitable stent coating apparatus (DES coater, in-house development of Biotronik). After reaching the intended layer weight of approx. 400 μg, the stent is dried in vacuo at room temperature and a second polymer layer of a PLGA-PEG block copolymer (Boehringer Ingelheim) is sprayed on it.

1. An implant with a base body at least partially comprised of a biocorrodible metallic material, whereby the material is such that it decomposes in an aqueous environment to form an alkaline product and whereby the base body has one or more of a coating and a cavity filling comprising a polymer matrix and at least one drug embedded in the polymer matrix, characterized in that at least one polymer of the polymer matrix and the at least one drug are coordinated so that the drug elution rate from the polymer matrix is increased at an elevated pH.

2. The implant according to claim 1, wherein the implant is a stent.

3. The implant according to claim 1, wherein the biocorrodible metallic material is a magnesium alloy.

4. The implant according to claim 1, wherein the drug elution rate from the polymer matrix is increased at a pH above 8.

5. Implant according to claim 1, wherein the drug elution rate from the polymer matrix is at least twice as high when the pH is greater than 8 as compared to the rate when the pH is at the physiological pH.

6. The implant according to claim 1, wherein the polymer has at least one functional group which shows a transition between an ionic charge state and a neutral charge state when there is an increase in pH.

7. The implant according to claim 6, wherein the functional group is selected from the group comprising a carboxylic acid function, an amine function and an amide function.

8. The implant according to claim 1, wherein the polymer matrix comprises a hydrogel.

9. The implant according to claim 8, wherein the hydrogel is selected from the group comprising a polymer based on acrylic acid, methacrylic acid, a derivative of acrylic acids, and methacrylic acid.

10. The implant according to claim 1, wherein the implant has an additional outer coating containing a degradable polymer.

11. The implant according to claim 1, wherein the drug is a prodrug embedded in the polymer matrix.

12. The implant according to claim 11, wherein the drug is affixed in the polymer matrix by chemical bonds cleaved by base catalysis.

13. The implant according to claim 1, wherein the drug is selected from the group comprising vasodilators, anti-inflammatories and pH regulating drugs.

14. The implant according to claim 1, wherein the drug is selected from the group comprising NO-eluting substances and bosentan, dipyridamol, dODN, endothelin receptor antagonists, calcium channel blockers, amlodipine, nifidipine and verapamil.

15. A stent comprising:
a base body at least partially comprised of a biocorrodible magnesium alloy that decomposes in an aqueous environment to form an alkaline product;
the base body having one or more of a coating and a cavity filling comprising a hydrogel;
at least one drug embedded in the hydrogel, the at least one drug and the hydrogel selected to result in the drug elution rate from the hydrogel being increased at an elevated pH; and,
an outer coating containing a degradable polymer.

16. A stent comprising:
a base body at least partially comprised of a biocorrodible magnesium alloy that decomposes in an aqueous environment to form an alkaline product;
the base body having one or more of a coating and a cavity filling comprising a hydrogel, the hydrogel selected from the group comprising a polymer based on acrylic acid, methacrylic acid, a derivative of acrylic acid, and methacrylic acid;
at least one drug embedded in the hydrogel, the at least one drug and the hydrogel selected to result in the drug elution rate from the hydrogel being increased at an elevated pH, the drug selected from the group comprising NO-eluting substances and bosentan, dipyridamol, dODN, endothelin receptor antagonists, calcium channel blockers, amlodipine, nifidipine and verapamil; and,
an outer coating containing a degradable polymer.
